(19) United States
(12) Patent Application Publication
(10) Pub. No.: US 2020/0229735 A1
(43) Pub. Date: Jul. 23, 2020

(54) MEASURING EXHALED NITRIC OXIDE WITH VARIABLE FLOW RATE

(71) Applicant: University of Southern California, Los Angeles, CA (US)

(72) Inventor: Patrick Muchmore, Hermosa Beach, CA (US)

(73) Assignee: University of Southern California, Los Angeles, CA (US)

(21) Appl. No.: 16/632,247

(22) PCT Filed: Jul. 16, 2018
(86) PCT No.: PCT/US2018/042316

§ 371 Date: Jan. 17, 2020

Related U.S. Application Data
(60) Provisional application No. 62/533,529, filed on Jul. 17, 2017.

(51) Int. Cl. A61B 5/08; A61B 5/087; A61B 5/00

(52) U.S. Cl. CPC A61B 5/082; A61B 5/0004; A61B 5/7239; A61B 5/087

(57) ABSTRACT

Asthma creates inflation of the epithelial cells in the airway of a patient. Inflammation causes epithelial cells to increase the production of nitric oxide far above the normally low levels. Therefore, clinicians can detect biomarkers of Asthma and other maladies by measuring the concentration of exhaled nitric oxide (“eNO”) for fractional exhaled nitric oxide (“FeNO”). However, current systems require the patient to exhale at a constant rate to estimate the concentration eNO. This rough approximation may under or overestimate the FeNO, which can cause misdiagnosis. Accordingly, disclosed are systems and methods to determine the amount of nitric oxide exhaled that compensate for a variable flow rate of exhaling, and do not assume a constant flow rate.

### STATEMENT REGARDING FEDERALLY SPONSORED RESEARCH OR DEVELOPMENT

This invention was made with government support under Grant No. ES022987 awarded by the National Institutes of Health. The government has certain rights in the invention.

### FIELD OF THE DISCLOSURE

The present invention is directed to methods for detecting concentration of nitric oxide in exhaled gases.

### BACKGROUND OF THE DISCLOSURE

The following description includes information that may be useful in understanding the present invention. It is not an admission that any of the information provided herein is prior art or relevant to the presently claimed invention, or that any publication specifically or implicitly referenced is prior art.

In the early 1990s, it was discovered the human respiratory system produces nitric oxide (NO) in sufficient quantity to measure in a straightforward manner. Early follow-up studies investigated associations with respiratory conditions, particularly asthma, and the fractional concentration of NO in exhaled breath (FeNO) has been recognized as a biomarker for this disease.

Measuring exhaled NO is noninvasive, affordable, and infinitely repeatable, endowing it with obvious clinical appeal. However, a significant impediment to widespread use of NO testing is the degree to which the measured concentration can be confounded by other factors. The most significant is the rate at which the subject exhales. This can be partially addressed by standardizing the exhalation rate (e.g., 50 ml/s in most guidelines); however, this limits the possible interpretations of the data, and other sources of variation, such as subject size, are unaccounted for.

Beginning in the late 1990s, as the severity of the problem became clear, a number of researchers explored more sophisticated modeling approaches as a way to account for non-clinical sources of variation in FeNO. Over the span of roughly a decade, researchers such as Dr. Steven George (of UC Irvine at the time) demonstrated that many qualitative features of exhaled NO can be accurately described by the partial differential equation (PDE) that results from imposing conservation of (NO) mass throughout the airway.

### SUMMARY OF THE PRESENT DISCLOSURE

Despite some progress being made in NO modeling, these advances have rarely been employed outside of a laboratory setting. Accordingly, more sophisticated dynamic modeling approaches have been developed.

Accordingly, a library of Java software routines have been developed to solve the relevant PDE, along with a collection of routines enabling Markov chain Monte Carlo (MCMC) based inference for the equation parameters. While the disclosed approach to inference is computationally demanding, with a sample of CHS data, the complete analysis, from raw data to formatted output, can be done in a matter of minutes (or less) on a typical PC.

Most existing approaches to sampling assume the subject is able to control their exhalation rate with significant precision. Even for healthy adults this may be difficult, and for other groups, such as young children, it may preclude FeNO testing entirely. The disclosed approach, on the other hand, uses measured flow data to continuously adjust the model; therefore, one can analyze data gathered at continuously varying flow rates. This offers the potential for the disclosed approach to enable FeNO testing for subjects unable to perform existing protocols.

Although the two-compartment model has proven useful for modeling many qualitative characteristics of exhaled NO, in some respects is only a gross approximation of reality. For instance, in Weibel's widely used model of the lung (12), the human airway consists of a series of branching passages and the number of branches in each generation grows exponentially as one progress deeper into the airway. If the airway shape, and specifically the cross sectional area, vary as one moves axially (which is implied by Weibel's model), solving the dynamic problem becomes more difficult.

Although adopting a more realistic airway model offers obvious appeal, it comes with some less obvious implications. Specifically, a fundamental quantity in the solution of equations is the Peclet number, which is defined to be the ratio of advective and diffusive velocities,

$$\frac{v ( t )}{d} ( 10 ) .$$

The advective velocity v(t) is the volumetric flow rate divided by the cross-sectional area. In a branching model, the cross-sectional area increases as one moves deeper into the airway (12), which implies the velocity is a function of both the time and position: v=v(t,z). As the cross-sectional are increases, the advective velocity, and hence the relative importance of advection, diminishes. Thus, although diffusion plays a modest role in the two-compartment model, it is likely to play a more significant role in more physiologically realistic models.

### EMBODIMENTS

### Embodiment 1

A system for determining a concentration of nitric oxide in exhaled breath, the system comprising:

a flow chamber;

a flow sensor connected to the flow chamber positioned to detect flow of gas inside the flow chamber;

a nitric oxide sensor positioned inside the flow chamber, the nitric oxide sensor positioned to detect a local nitric oxide concentration of gas inside the flow chamber;

a memory containing machine readable medium comprising machine executable code having stored thereon instructions for performing a method of determining an exhaled concentration of nitric oxide; and

a control system coupled to the memory, the control system configured to execute the machine executable code to cause the processor to:
receive, from the flow sensor, flow rate data points that include data related to a flow rate and a time stamp corresponding to the flow rate;
receive, from the nitric oxide sensor, concentration data points that comprise data related to a local nitric oxide concentration and a time stamp corresponding to the local nitric oxide concentration;
associate, by the control system, the flow rate data points and the concentration data points that both have a time stamp indicating they were taken within close or at same temporal proximity;
store, by the control system, associated flow rate data points and concentration data points for an exhale event; and
determine, by the control system, an indication of an exhaled nitric oxide concentration based on at least a subset of the stored associated flow rate data points and concentration data points for the exhale event, wherein the subset of stored associated flow rate data points comprises data related to different flow rates.

### Embodiment 2

The system of embodiment 1, wherein the step of determining the indication of an exhaled nitric oxide concentration is performed using a differential equation to model the flow rate.

### Embodiment 3

The system of embodiment 2, wherein the differential equation is an advection-diffusion-reaction partial differential equation.

### Embodiment 4

The system of embodiment 1, wherein the flow rate data points by the flow sensor is output to a low pass filter.

### Embodiment 5

The system of embodiment 3, wherein parameter estimates of the advection-diffusion-reaction partial differential equation are run by the control system using a Markov Chain.

### Embodiment 6

The system of embodiment 1, wherein the flow sensor is selected from at least one of: a pressure differential sensor, an ultrasonic flow meter, an optical flow sensor, and a mechanical flow sensor.

### Embodiment 7

The system of embodiment 1, wherein the nitric oxide sensor comprises an electrochemical sensor.

### Embodiment 8

The system of embodiment 1, wherein the control system is embedded in a remote server or a database.

### Embodiment 9

The system of embodiment 1, wherein the flow chamber comprises a mouthpiece.

### Embodiment 10

A system for determining a concentration nitric oxide in exhaled breath in a patient, the system comprising:

a device that measures and transmits at least two output signals associated with the patient;

a computing device configured to receive, record, store, and analyze the at least two output signals from the device to generate the patient's concentration of nitric oxide in exhaled breath; and

a graphical user interface on the computing device that allows a user to view and customize options for monitoring the concentration of nitric oxide in exhaled breath,

wherein the device, the at least one remote device, and the computing device are communicatively connected to each other via a communications network,

wherein the device comprises a flow chamber, a flow sensor and a nitric oxide sensor,

wherein the at least two outputs signal comprises: (i) flow rate and a time stamp corresponding to the flow rate and (ii) a local nitric oxide concentration and a time stamp corresponding to the local nitric oxide concentration,

wherein the computing device receives, records, and stores associated flow rate data points and concentration data points for an exhale event of the patient, and

wherein the computing device is configured to determine an indication of an exhaled nitric oxide concentration based on at least a subset of the stored associated flow rate data points and concentration data points for the exhale event, wherein the subset of stored associated flow rate data points comprises data related to different flow rates.

### Embodiment 11

The system of embodiment 10, wherein the system further comprises a hosted server that is (i) configured to store and analyze the at least two output signals, and (ii) connected to the device, the at least one remote device, and the computing device via the communications network.

### Embodiment 12

The system of embodiment 11, wherein the hosted server is further configured to measure, store, and analyze the at least two output signals associated with a certain patient and dynamically aggregate and analyze the exhale event of the patient with at least two output signals to determine the concentration of nitric oxide in exhaled breath associated with the patient.

### Embodiment 13

The system of embodiment 10, wherein the device is configured to send an alarm or a notification to the computing device or a healthcare professional based on a level of the concentration of nitric oxide in exhaled breath.

### Embodiment 14

The system of embodiment 10, wherein the device is configured to detect a health event based on the at least two output signals and send an alert to at least one of following: the device, the computing device, or a healthcare professional.

### Embodiment 15

The system of embodiment 10, wherein the step of determining the indication of an exhaled nitric oxide concentration is performed using a differential equation to model the flow rate.

### Embodiment 16

The system of embodiment 15, wherein the differential equation is an advection-diffusion-reaction partial differential equation.

### Embodiment 17

The system of embodiment 10, wherein the flow rate data points by the flow sensor is output to a low pass filter.

### Embodiment 18

The system of embodiment 16, wherein parameter estimates of the advection-diffusion-reaction partial differential equation are run by the control system using a Markov Chain.

### Embodiment 19

The system of embodiment 10, wherein the flow sensor is selected from at least one of: a pressure differential sensor, an ultrasonic flow meter, an optical flow sensor, and a mechanical flow sensor.

### Embodiment 20

The system of embodiment 10, wherein the nitric oxide sensor comprises an electrochemical sensor.

### Embodiment 21

The system of embodiment 10, wherein the flow chamber comprises a mouthpiece.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts, in accordance with various embodiments of the present invention, a perspective view of a system for determining an amount of nitric oxide using a variable flow rate;

FIG. 2 depicts, in accordance with various embodiments of the present invention, a flow chart illustrating a process for determining an amount of nitric oxide using a variable flow rate;

FIG. 3 depicts, in accordance with various embodiments of the present invention, a diagram of an airway modeled for equations disclosure herein;

FIG. 4 depicts, in accordance with various embodiments of the present invention, a graph illustrating raw and filtered flow data;

FIG. 5 depicts, in accordance with various embodiments of the present invention, a graph illustrating simulated eNO data and filtered flow data;

FIG. 6 depicts, in accordance with various embodiments of the present invention, graphs illustrating observed and estimated eNO data;

FIG. 7 depicts, in accordance with various embodiments of the present invention, a graphs illustrating experimental results;

FIG. 8 depicts, in accordance with various embodiments of the present invention, graphs illustrating observed and estimated eNO data;

FIG. 9 depicts, in accordance with various embodiments of the present invention, graphs illustrating CaNO, DawNO, and JawNO data; and

FIG. 10 depicts, in accordance with various embodiments of the present invention, a diagram of an airway modeled for equations disclosure herein.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION OF THE PRESENT DISCLOSURE

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in” and “on.” The phrase “in one implementation,” as used herein does not necessarily refer to the same implementation

The term “coupled” means at least either a direct electrical connection between the connected items or an indirect connection through one or more passive or active intermediary devices. The term “circuit” means at least either a single component or a multiplicity of components, either active and/or passive, that are coupled together to provide a desired function. The term “signal” as used herein may include any meanings as may be understood by those of ordinary skill in the art, including at least an electric or magnetic representation of current, voltage, charge, temperature, data or a state of one or more memory locations as expressed on one or more transmission mediums, and generally capable of being transmitted, received, stored, compared, combined or otherwise manipulated in any equivalent manner.

Terms such as “providing,” “processing,” “supplying,” “determining,” “calculating” or the like may refer at least to an action of a computer system, computer program, signal processor, logic or alternative analog or digital electronic device that may be transformative of signals represented as physical quantities, whether automatically or manually initiated.

A “computer,” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a cloud, a super computer, a personal computer, a laptop computer, a palmtop computer, a mobile device, a tablet computer, a notebook computer, a desktop computer, a workstation computer, a server, or the like, or an array of processors, microprocessors, central processing units, general purpose computers, super computers, personal computers, laptop computers, palmtop computers, mobile devices, tablet computers, notebook computers, desktop computers, workstation computers, servers, or the like.

A “server,” as used in this disclosure, means any combination of software and/or hardware, including at least one application and/or at least one computer to perform services for connected clients as part of a client-server architecture. The at least one server application may include, but is not limited to, for example, an application program that can accept connections to service requests from clients by sending back responses to the clients. The server may be configured to run the at least one application, often under heavy workloads, unattended, for extended periods of time with minimal human direction. The server may include a plurality of computers configured, with the at least one application being divided among the computers depending upon the workload. For example, under light loading, the at least one application can run on a single computer. However, under heavy loading, multiple computers may be required to run the at least one application. The server, or any if its computers, may also be used as a workstation.

A “database,” as used in this disclosure, means any combination of software and/or hardware, including at least one application and/or at least one computer. The database may include a structured collection of records or data organized according to a database model, such as, for example, but not limited to at least one of a relational model, a hierarchical model, a network model or the like. The database may include a database management system application (DBMS) as is known in the art. The at least one application may include, but is not limited to, for example, an application program that can accept connections to service requests from clients by sending back responses to the clients. The database may be configured to run the at least one application, often under heavy workloads, unattended, for extended periods of time with minimal human direction.

A “communications network,” as used in this disclosure, means a wired and/or wireless medium that conveys data or information between at least two points. The wired or wireless medium may include, for example, a metallic conductor link, a radio frequency (RF) communication link, an Infrared (IR) communication link, telecommunications networks, an optical communication link, internet (wireless and wired) or the like, without limitation. The RF communication link may include, for example, WiFi, WiMAX, IEEE 802.11, DECT, 0G, 1G, 2G, 3G, 4G, 5G or future cellular standards, Bluetooth, Bluetooth Low Energy, NFC, ultrasound, induction, laser (or similar optical transmission) and the like.

A “computer-readable storage medium” or “machine readable medium,” as used in this disclosure, means any medium that participates in providing data (for example, instructions) which may be read by a computer. Such a medium may take many forms, including non-volatile media, volatile media, and transmission media. Non-volatile media may include, for example, optical or magnetic disks, flash memory, and other persistent memory. Volatile media may include dynamic random access memory (DRAM). Transmission media may include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium or machine readable medium may include a “Cloud,” which includes a distribution of files across multiple (e.g., thousands of) memory caches on multiple (e.g., thousands of) computers.

Various forms of computer readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

A “network,” as used in this disclosure means, but is not limited to, for example, at least one of a local area network (LAN), a wide area network (WAN), a metropolitan area network (MAN), a personal area network (PAN), a campus area network, a corporate area network, a global area network (GAN), a broadband area network (BAN), a cellular network, the Internet, the cloud network, or the like, or any combination of the foregoing, any of which may be configured to communicate data via a wireless and/or a wired communication medium. These networks may run a variety of protocols not limited to TCP/IP, IRC, SSL, TLS, UDP, or HTTP.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

Asthma creates inflation of the epithelial cells in the airway of a patient which causes epithelial cells to increase the production of nitric oxide far above the normally low levels. Therefore, clinicians can detect biomarkers of Asthma and other maladies by measuring the concentration of exhaled nitric oxide (“eNO”) for fractional exhaled nitric oxide (“FeNO”). However, current systems require the patient to exhale at a constant rate to estimate the concentration eNO. This rough approximation may under or overestimate the FeNO which can cause misdiagnosis.

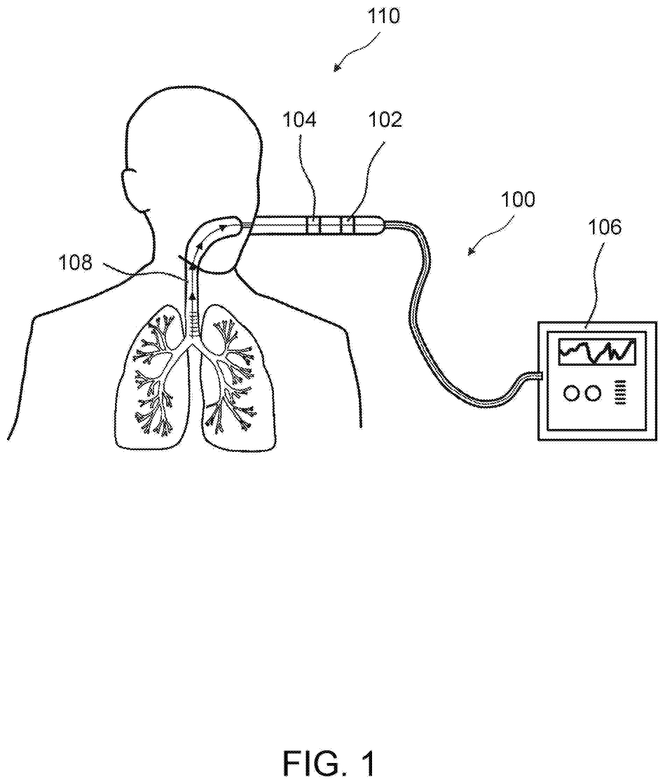

Accordingly, disclosed are systems and methods to determine the amount of nitric oxide exhaled that compensate for a variable flow rate of exhaling, and do not assume a constant flow rate. For instance, FIG. 1 illustrates an overview of an example system 100 utilized to determine the amount of nitric oxide exhaled by a patient. The system 100 includes a mouthpiece 110 that a patient may hold up to their airway (e.g., throat) 108. The mouthpiece 100 includes a flow sensor 104, a nitric oxide sensor 102, and a computing device 106, all of which may be connected to each other via a tube, electric wire, or another communications link. The patient may exhale into the mouthpiece 110, and the flow sensor 104 and the nitric oxide sensor 102 may record readings during the exhalation at various times. In some examples, the flow sensor 104 and the nitric oxide sensor 102 may be located together to take flow and NO readings from the same position. In an embodiment of the present disclosure, the flow sensor 104 and the nitric oxide sensor 102 may be located separately in different locations.

The readings from the flow sensor 104 and NO sensor 102 may be sent electronically or by other means to a computing device 106 that records the readings, stores them in memory, sends them over to a server for analysis, or analyzes them locally on a local processor or other control system.

The flow sensor 104 may be any suitable flow sensor for measuring the flow of air exhaled. This may include a restriction type flow sensor that measures the pressure differential across the restriction. Other examples include a mechanical based flow sensor, anemometers, an optical flow sensor, or an ultrasonic flow meter. In some examples, the system 100 may include a heater, or a dehumidifier.

The nitric oxide sensor 102 may be an electrochemical sensor or other suitable sensor for detecting instantaneous values of nitric oxide. For instance, the NO sensor 102 may be configured to detect NO concentrations in the parts-per-billion range. In some examples, a chemiluminescence analyzer may be utilized available from Signal USA at http://www.k2bw.com/chemiluminescence.htm. In other examples, surface acoustic wave sensors may be utilized described in “A Room Temperature Nitric Oxide Gas Sensor Based on aCopper-Ion-Doped Polyaniline/Tungsten Oxide Nanocomposite” by Wang et al., which is incorporated by reference in its entirety.

The computing device 106 may be any suitable computing device that could be a personal computer, mobile device, or other device. In some examples, the computing device 106 may be linked to a remote server and/or a database for processing the data, and will temporarily store the data to transmit after or during the test to a remote server. Other various configurations of sending and processing of the data at various locations could be described and are therefore incorporated within the scope of this disclosure.

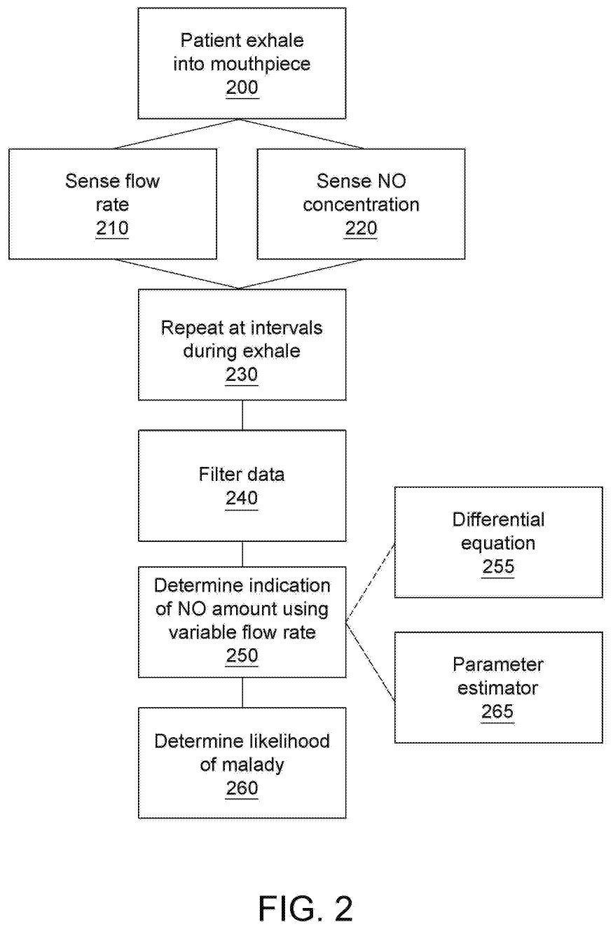

FIG. 2 is a flow chart illustrating an example process for identifying whether a patient has a malady. First, a patient exhales into the mouthpiece of the system 100 (S200). The flow sensor(s) 102 will sense the flow rate (S210) and the NO sensors (s) 104 will detect the nitric oxide concentration (S220). This will be repeated at various times during exhalation (S230), so that the system 100 simultaneously or in close temporal proximity records the flow rate (S210) and NO concentration (S220) at intervals during the breath cycle.

Then, output from the sensors including the detected flow rate (S210) and NO levels (S220) at various times during the exhalation cycle will be filtered (S240) or otherwise processed. In some examples, this may include a low pass filter, or data reduction techniques. Also, the quantity of data points may be reduced to lower the computational load during consistent flow rate portions of the breathing cycle (for instance in the middle of the exhalation). In some examples, the flow rate (S210) and NO level (S220) will be detect ever cycle, twice a second, at 4 Hz, or other suitable times based on the responsiveness of the sensors and the needs of the detection algorithm.

Next, the filtered and processed data will be analyzed to determine a FeNO or other metric indicating an amount of nitric oxide exhaled (S250) based on the different flow rates detected (S250). This may be performed by a variety of models (S255) that compensate for a variable flow rate. For instance, in some examples, the model (S255) may be based on an advection-diffusion reaction differential equation (S255). Using these types of models 255, various parameter estimation methods (S265) may be implemented that are detailed below.

Then, in some embodiments, once a determination is made of the FeNO a correlation may be made or indication of whether the patient has a malady such as asthma (S260). For instance, in some examples, a threshold level of PPB or a tiered score based on several different thresholds may give the patient an estimation of the likelihood they have asthma (S260) or some other malady. This indication may be output as a positive or negative, a quantitative score, an alert to a physician to follow up or other indication.

### The Dynamic Two Compartment Model

One model (S255) for NO exchange divides the respiratory system into two parts, known as the airway and alveolar compartments. In its simplest form, the two-compartment airway is assumed to be a cylinder with fixed dimensions. During exhalation, air enters this cylinder from the alveolar compartment, passes through the airway, and exits at the mouth. The airway cylinder is lined with tissue that is assumed to be NO permeable, with a constant coefficient of permeability. The tissue NO concentration is also assumed to be constant; therefore, depending on the relative concentrations, the airway tissue serves as either an infinite source or sink for airway NO.

Air entering from the alveolar region is the other source of exhaled NO. Like the airway tissue, the NO concentration and permeability of the alveolar tissue are assumed constant. Unlike the airway, the volume of the alveolar region may vary (at least as described in (11)). However, at any moment in time the concentration of NO is assumed to be constant throughout this region, i.e. it is “perfectly mixed”. In some examples, the two-compartment model also incorporate a time varying alveolar concentration; in practice it is often assumed to be constant on short (minutes) time scales.

### The Governing Equation

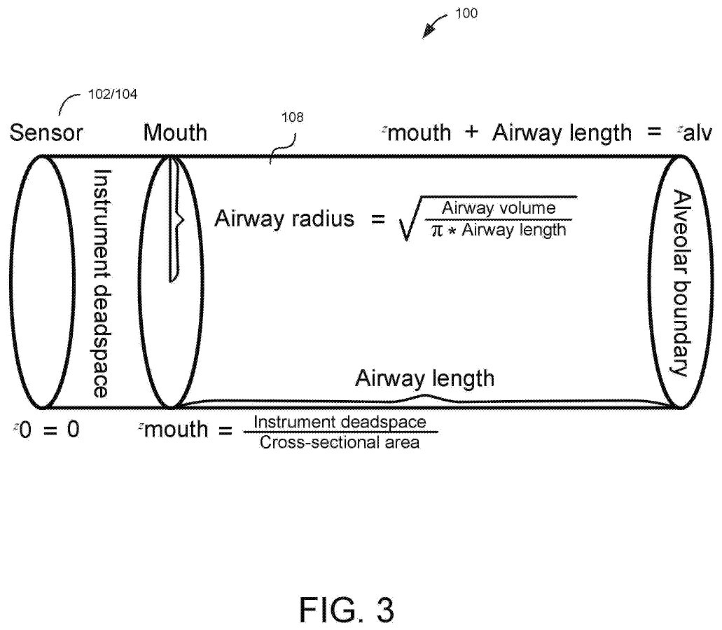

One embodiments of a model (S255) is a variant of the two-compartment abstraction described in (11), the primary difference being assumptions are made regarding flow rate which can be variable. The alveolar compartment is assumed to have a constant NO concentration through both time and space. During exhalation, air exits the alveolar compartment and passes into the airway, which corresponds to air entering the airway through the right hand boundary of FIG. 3.

As air passes through the lumen, the airway wall will act as either a source or sink of NO, depending on the relative concentration between the airway lumen and wall. The biological airway ends at the mouth; however, the instrument dead space volume extends the “airway” cylinder. In this region, the airway “wall” is assumed to be impermeable to NO; otherwise, it is modeled in the same manner as the rest of the airway. In this regard, the model is equivalent to a cylindrical model with a piecewise variable airway wall concentration.

### Cylindrical Airway Model

In some examples, the dynamics of NO in the airway are assumed can be modeled (S255) by the advection-diffusion-reaction (ADR) partial differential equation:

$$\begin{matrix}Equation 1. Advection \text{-} Diffussion \text{-} reaction ( ADR ) \\ partial differential equation & \\ \frac{\partial c}{\partial t} = - v ( t ) \frac{\partial c}{\partial z} + d \frac{{\partial }^{2} c}{\partial {z}^{2}} + \frac{2 p}{r} [ {c}_{w} - c ( t , z ) ] , & ( 1 )\end{matrix}$$

Where c(t,z) is the NO concentration at time t and position z, in ppb,

v(t) is the linear flow rate, in

$$\frac{cm}{s} ,$$

d is the diffusivity of NO in air, in

$$\frac{{cm}^{2}}{s} ,$$

p is the permeability of airway wall tissue to NO, in

$$\frac{cm}{s} ,$$

r is the airway radius in cm, and

c_{w} is concentration of NO in the airway wall tissue, in ppb.

In order, the three quantities on the right hand side are known as the advection, diffusion, and reaction terms (respectively). In this context the last quantity is also known as the source term, as it represents another source of NO, namely, the airway wall. The contribution from the airway wall is proportional to the difference in concentration between the airway wall and the airway lumen. Multiplying the coefficient through yields

$$\frac{2 p}{r} {c}_{w} - \frac{2 p}{r} c ( t , z ) .$$

The first term

$$\frac{2 p}{r} {c}_{w} ,$$

is constant, and hence referred to as the constant source. The contribution from the second term is proportional to the concentration c(t,z); therefore, the quantity

$$\frac{2 p}{r}$$

is called the proportional source term.

In the cylinder illustrated in FIG. 3, air flows left-to-right (→) during inhalation, and right-to-left (←) during exhalation. The alveolar concentration corresponds to the concentration at the right boundary, c(t, zₐₗᵥ). During exhalation, this boundary value is assumed to be constant, and corresponds to the alveolar concentration parameter. A simple derivation of equation (1) is given in the appendix, along with an introduction to the “conservative form” representation, see e.g. (7).

In some examples, the alveolar concentration parameter is defined as equivalent to the parameter in the basic two-compartment model (S255) (3). The constant source and proportional source terms are pointwise equivalents of the J'aw_{NO} and Daw_{NO} and parameters (respectively). When the airway volume is assumed to be constant (as in the two-compartment model), the pointwise parameters can be transformed into their global equivalents simply by multiplying by the airway volume, that is =airway volume×constant source and Daw_{NO}=airway volume×proportional source.

### Examples

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Parameter Estimation

Accordingly, once the model (S255) is selected, the parameters must be estimated (S265). In some examples the system 100 may use a Bayesian approach to inference in order to characterize the posterior distribution, generically denoted ƒ(θ|x) for parameters θ and data x. Using Bayes rule, the posterior can be expressed in terms of the likelihood ƒ(x|θ) and a prior distribution ƒ(θ). In some examples, the unnormalized posterior is sufficient, simplifying the relationship between to the posterior, likelihood, and prior to the proportionality ƒθ|x)∝ƒ(x|θ)ƒ(θ).

There are numerous possible parameterizations of the model (S255); in the subsequent examples, the parameters of interest θ are assumed to be the constant source, the proportional source, and the alveolar concentration (recalling that the first two can easily be transformed into J'aw_{NO} and Daw_{NO}). The choice of parameterization can have a significant impact on both the interpretability of the parameter estimates (S265) and the efficiency of the calculation. Some of the tradeoffs inherent in this choice of parameterization, along with possible alternatives, are disclosed herein.

For the model (S255) in general, the data x corresponds to the observed NO concentrations at the sensor (z=0), measured over some continuous time interval. The data used in the examples comes from a multiple flow study, so the data consists of multiple observation windows. Each maneuver produces a times series of 100 s-1000 s of NO measurements; therefore, the measured NO values are denoted xᵢⱼ, where i indexes the maneuvers, and j indexes the time points within a maneuver.

The concentration predicted by the model (S255) based on Equation (1) at the sensor 104/102 is denoted cᵢⱼ: =c(tⱼ, 0), where tⱼ is the time corresponding to index j. To formulate a likelihood, one may assume that by fixing θ and solving the corresponding model (S255) equation, the model (S255) solution can be used to calculate the density of the observed data. If we further assume the xᵢⱼ share a common parametric conditional density function, and that conditional on the model solutions cᵢⱼ the xᵢⱼ are independent, then the likelihood can be written as:

$$f ( x \vert θ ) = {Π}_{i} {Π}_{j} f ( {x}_{ij} \vert {c}_{ij} ) , \qquad Equation 2. Likelihood$$

where θ appears implicitly on the right hand side via the model solution cᵢⱼ.

When the likelihood (2) is combined with a prior ƒ(θ), the (unnormalized) posterior can be easily calculated for any particular set of parameters θ. To efficiently explore the posterior distribution we employ a Metropolis-Hastings style MCMC algorithm, which generically proceeds as follows:

Select an initial value θ and calculate the likelihood ƒ(θ|x).

Propose a new value θ′ using a transition kernel q(θ→θ′), and calculate the likelihood ƒ(θ′|x).

Accept the proposed value with probability

$$min [ 1 , \frac{f ( x \vert θ′ ) f ( θ′ ) q ( θ′ \to θ )}{f ( x \vert θ ) f ( θ ) q ( θ \to θ′ )} ] .$$

If proposal is accepted set θ=θ′ and ƒ(θ|x)=ƒ(θ′|x) then return to 1; otherwise, return directly to 1.

The choice of transition kernel q can have a significant impact on the efficiency of this type of algorithm. Finding an optimal q can be difficult; however, there are a number of more recent MCMC algorithms, which incorporate an “adaptive” transition distribution (9). To better account for variability in the posterior across individuals, the adaptive Metropolis algorithm of (4) is employed to automatically calibrate the proposal distribution against the target. This has the dual benefit of both increasing the efficiency of our sampler, while also simplifying the user experience by largely automating the choice of transition kernel.

### Simulating the Dynamic Model

One approach to estimation is predicated upon repeated simulation of the underlying physical model. The method of lines (MOL) technique is applied to equation (1), wherein the spatial (z) variable is discretized using finite differences: upwind for the advective term, and centered for the diffusive. The time variable remains continuous, and the resultant semi-discrete problem can be solved numerically using routines developed for systems of ordinary differential equations (5, 8).

### Flow Rate Data Preprocessing

Modern integration routines, such as the Dormand-Prince (1) based method we employ, calculate running error estimates that can be used to adaptively vary the time integration step size. Although this is a useful feature, one consequence is that the solution is approximated at irregularly spaced intervals. The velocity v(t) is treated as an (observed) input in the disclosed model (S255), and it is sampled concurrently with NO. These values must be interpolated to estimate v(t) at the adaptively chosen times, and the manner in which this is done has a major impact on the efficiency of the routine.

Sharp changes in the concentration gradient are more difficult to resolve numerically, and thus require shorter time steps be taken. Because the concentration is flow dependent, sharp changes in the flow rate can lead to sharp changes in the concentration gradient, leading to a significant increase in computation time. The flow measurements are taken at discrete times, and equal one of a discrete set of possible values. Naively interpolating these points can lead to spurious high frequency oscillations, which significantly increases the number of steps taken, and hence the computation time.

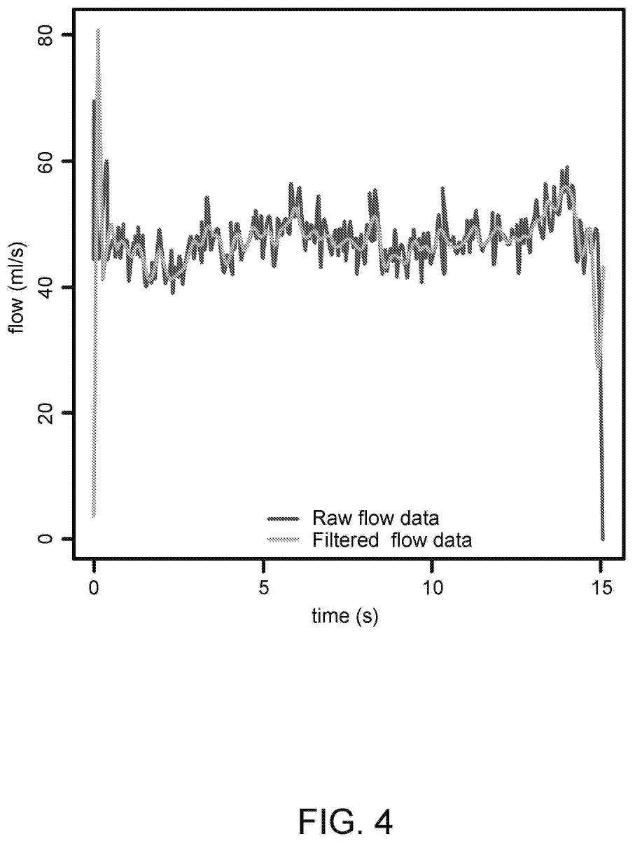

The darker line in FIG. 4 illustrates this phenomena, for what is nominally a sustained exhalation at 50 ml/s. As FIG. 4 shows, throughout the maneuver the flow rate measurements vary over a range of 10-15 ml/s. At times, the measured rate can oscillate rapidly over a discrete range of values, leading to a significant slowdown in the integration routine.

### Observed and Filtered Flow

To eliminate these oscillations, the data is run through a low-pass frequency filter.

Because the data is analyzed “offline”, i.e. after all of it has been collected, two pass (forward-backward) filtering is employed. The darker line in FIG. 4 illustrates the result from applying a fourth order Butterworth (6) filter to the raw signal, with a low-pass frequency threshold of 5 Hz. As FIG. 4 shows, filtering the signal in this manner retains the gross features, such as the spikes at the beginning, while eliminating the rapid oscillations later on.

### Numerical Integration and Simulation.

To solve equation numerically, the spatial (z) derivatives are replaced with finite difference approximations based on Taylor series expansions. For the diffusive term, a centered three term Taylor series approximation is employed,

$$\frac{{\partial }^{2} c}{\partial {z}^{2}} \approx \frac{c ( t , z - Δ z ) - 2 c ( t , z ) + c ( t , z + Δ z )}{{( Δ z )}^{2}} .$$

For the advective term, a biased 4 term Taylor series approximation is employed. The direction of the bias is determined by the direction of flow, as dictated by the sign of v(t). Specifically, the approximation is oriented with an “upwind” bias; two of the terms in the approximation are chosen on the side from which the flow originates, and only one is chosen from the opposite side.

For example, with flow moving in the positive direction (v(t)>0), the approximation may be defined as

$$\frac{\partial c}{\partial z} \approx \frac{c ( t , z - 2 Δ z ) - 6 c ( t , z - Δ z ) + 3 c ( t , z ) + 2 c ( t , z + Δ z )}{6 Δ z} .$$

An upwind discretization is chosen because centered discretization for advection can lead to spurious oscillations in the numerical approximation. A completely one-sided discretization can prevent oscillations; however, despite the formal order of the Taylor series, such an approximation will always have first order accuracy (5). By employing a two-sided, but biased, discretization, higher order accuracy can be achieved, while minimizing the potential for oscillatory solutions.

Replacing the spatial derivatives with their finite difference approximations yields a large system of ordinary differential equations. When combined with appropriate boundary and initial conditions (discussed in the appendix), “off-the-shelf” software can be used to perform the time integration (5). As illustrated in FIG. 3, the position of the sensor is defined to be the origin, z=0. Therefore, the solution at this point over time corresponds to the model prediction of eNO measured throughout the maneuver; informally e\hat{N}O=ĉ(t, 0), where ĉ(t, 0) is a numerical approximation of the true solution c(t, 0) to equation at time t.

In this framework, “simulating” the model largely consists of calculating a series of approximate solutions ĉ(t₀, 0), ĉ(t₁, 0), ĉ(tₙ, 0), where exhalation begins at t₀ and ends at tₙ. The approximate solutions depend on the airway parameter values; in the following calculations=2, =5, =800, and the airway volume is assumed to be 125 ml (implying the constant source and proportional source terms are 800/125=6.4 and 5/125=0.04, respectively).

Approximating the solution of equation also requires specifying the function v(t). In a sense, v(t)“drives” the solution, because it is the only term on the right hand side that varies with time. Rather than attempting to recreate such a complicated process from scratch, to approximate the function v(t) one may employ real flow data (gathered as part of the CHS). The flow data is filtered (as previously described), and at time t the approximation \hat{v}(t) is determined by interpolating the filtered flow data.

### Filtered Flow and Simulated eNO

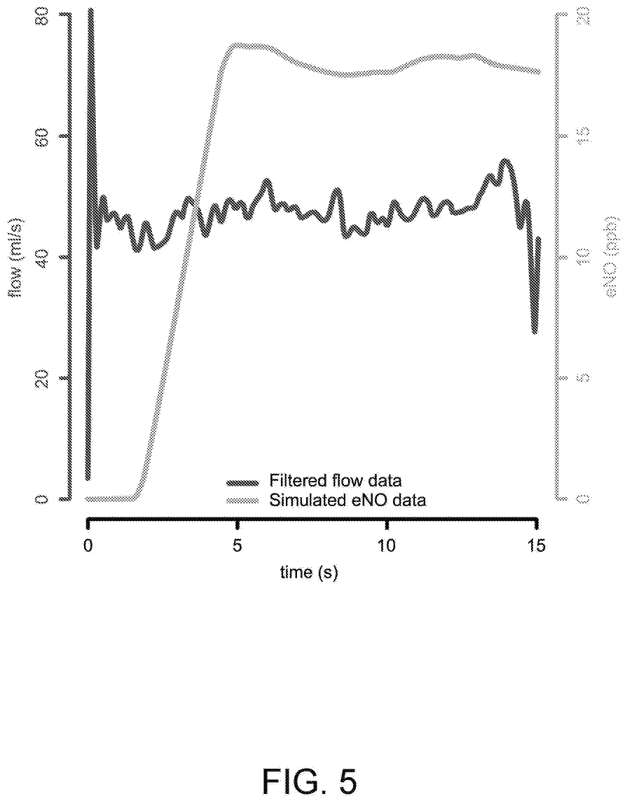

When the function \hat{v}(t) is combined with the parameter values specified above, numerical integration routines can be employed to calculate the sequence of approximations ĉ(tᵢ, 0), as illustrated in FIG. 5. The darker line is the same filtered flow data as shown in FIG. 5. The lighter line illustrates the predicted concentration at the sensor throughout the exhalation (synchronized with the flow, so the time scale is shared). The approximation ĉ(tᵢ, 0) is calculated at a few hundred time steps tᵢ; the integration routine automatically and adaptively estimates an optimal time step size, so the precise number of steps can vary across simulations.

Simulated eNO, with Noise Added and Filtering

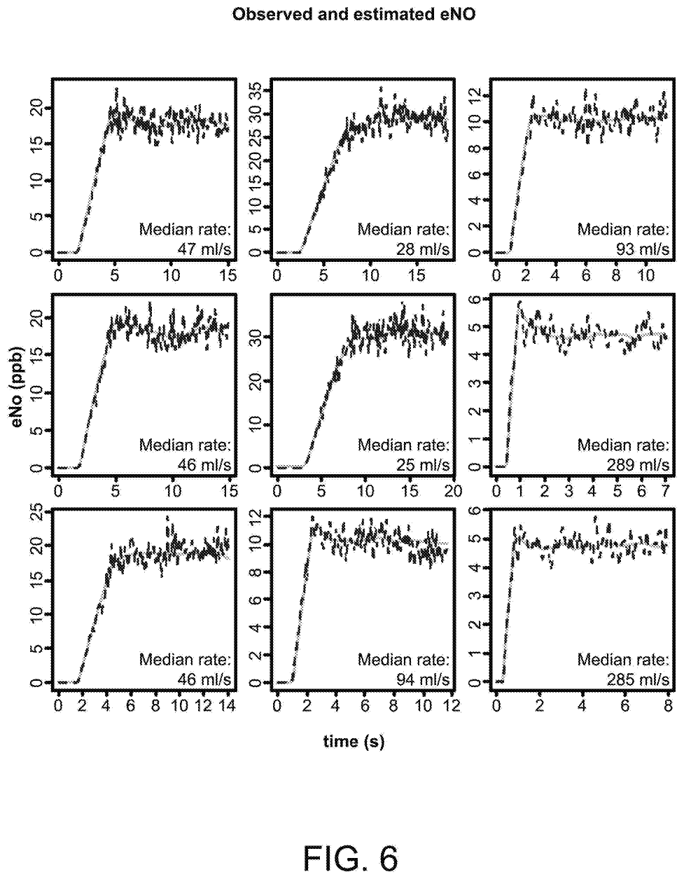

The deterministic PDE model leading to the lighter colored curve is capable of accurately describing many of the qualitative features of exhaled nitric oxide (10); however, the model is not perfect, and there will inevitably be other sources of variation. To account for this residual variation, independent errors are added to the deterministic solution. The lighter curve in the top left panel of FIG. 6 is identical to the eNO curve in FIG. 5, while the dashed darker line is the result of adding independent and identically distributed log normal errors to the deterministic solution. The other 8 panels in FIG. 6 are the result of repeating this process with the remaining flow profiles for this subject.

### Inference Example and Simulation Study

By treating the darker lines in FIG. 6 as real data, one can use the previously described MCMC inference machinery to estimate the values of CA_{NO}, Daw_{NO}, and J'aw_{NO}. Of course, because the data was generated using known parameter values, the expectation is the estimates will be consistent with those values.

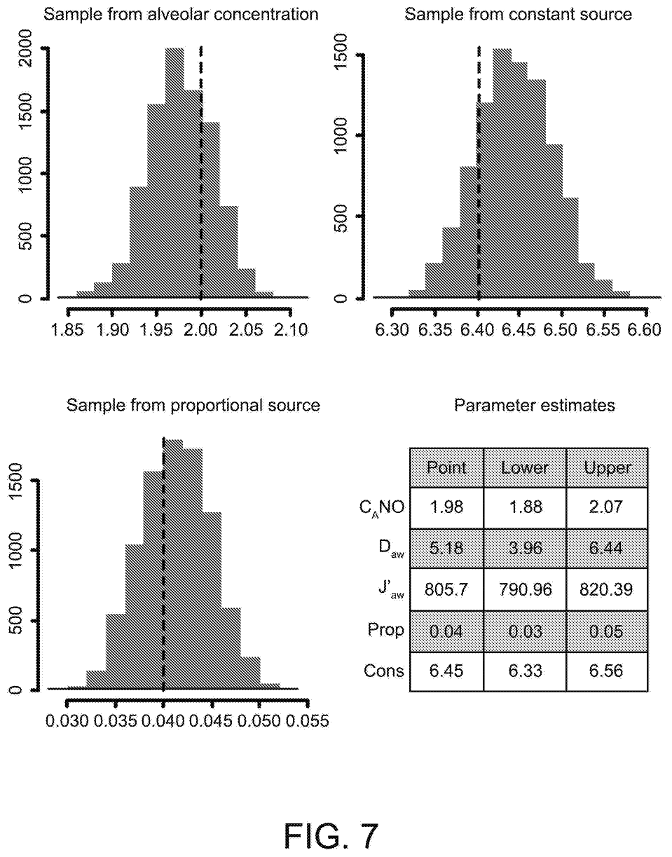

FIG. 7 illustrates an example of the parameter estimates generated by running the estimation routine using the simulated data as input. The Markov chain was run for 10,000 steps, and the acceptance rate was roughly 25%. The first 10% of the chain was discarded as “burn-in”, and the remainder used to generate the plots and table in FIG. 7.

As expected, the credible intervals for each of the three parameters contain the value used to generate the data. Both CA_{NO} and J'aw_{NO} appear to be estimated with significant precision, less so for Daw_{NO}. To quantify this precision, the estimation procedure used to generate FIG. 7 was repeated for a sample of 28 individuals from the CHS. To select these individuals, the CHS subjects were stratified based on their FeNO₅₀ measure: very low (<10 ppb), low (<25 ppb), intermediate (25-50 ppb), and high (>50 ppb), and 7 subjects were randomly chosen from each category.

The first row of Table 1 below provides the means and standard errors of the parameter estimates based on the stratified random sample. While the mean of the estimates for is almost exactly the true value, there are small biases in the estimates Daw_{NO} and J'aw_{NO}. These biases are the result of the frequency-filtering step applied to the NO data. Before filtering, the data is log transformed; however, a large number of measured NO values are zero. These zero values must be perturbed in some fashion before the log transformation, and this perturbation manifests itself as small biases in the parameter estimates. _

TABLE 1
Simulation study parameter estimates

| Error model | CA_{NO} | s. [symbol] . | Daw_{NO} | s. [symbol] . | J [symbol] _{NO} | s. [symbol] . |
|---|---|---|---|---|---|---|
| lognormal | 1.99 | 0.047 | 5.50 | 0.76 | 806.95 | 9.90 |
| normal | 2.00 | 0.053 | 5.02 | 0.48 | 800.37 | 6.79 |
| true values | 2 | | 5 | | 800 | |

[symbol]

indicates data missing or illegible when filed

If the log normal error model is replaced with a regular normal error model, the data does not need to be log transformed before filtering, eliminating the potential bias. To demonstrate this, the simulation study was repeated using the same 28 individuals, this time with residual errors that were normally distributed. The second row of table displays the corresponding parameter estimates, which do not display any evidence of the bias present in the log normal case.

Because the variable of interest is a concentration, by definition it should be non-negative. In this respect, the log normal distribution is a natural choice for modeling the residuals. Assuming a log normal error model also implies that measurements at, or very near, zero are subject to very little error; however, experience has shown this is often not the case.

Measurement errors often occur during regions of rapid change in eNO concentration, including within the first few seconds when the measured value initially deviates from zero. Assuming a normal error model more accurately captures the variability that occurs early in a maneuver, perhaps at the expense of being a less plausible model after the concentration has completed its initial ascent. The estimates in table are broadly similar for the two models, and in that regard, the choice of distribution is not crucial.

### Application to Multiple Flow Data

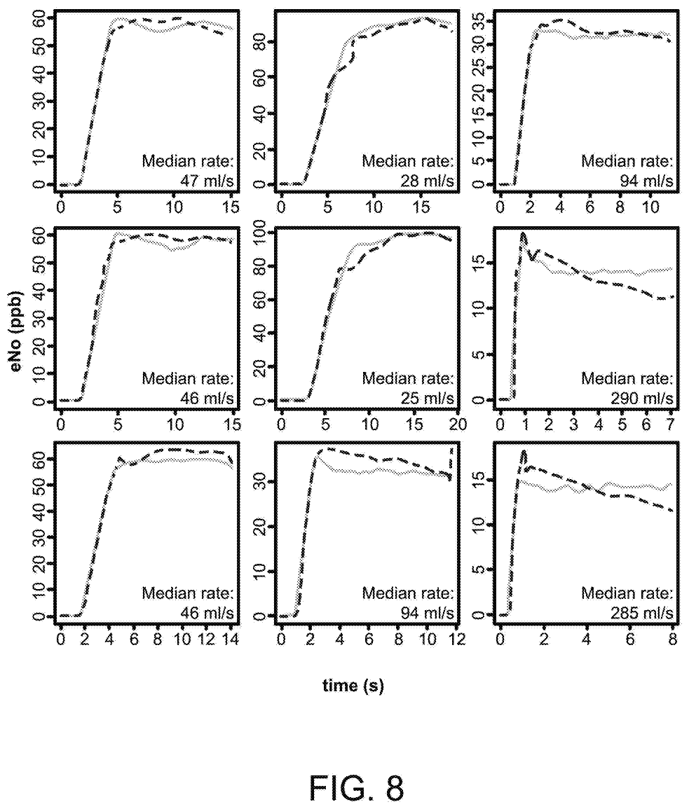

Having demonstrated that the dynamic model can consistently recover parameter estimates from simulated data, the model can be applied to real data using the same subject, and hence flow data, as before. The dashed lines in FIG. 8 are the actual, filtered, NO data gathered during the CHS. It is worth noting the observed NO values are significantly higher than the simulated profiles in FIG. 6.

The simulated data may also appear “noisier” than the observed. This is partially due to the fact that this subject was chosen for illustrative purposes because their data is “clean”. However, these figures also illustrate that sigma remains a crude proxy for other, systematic, sources of variation. Some possible extensions to the model that may better account for these sources are described in the discussion.

### Model Predictions and Parameter Estimates

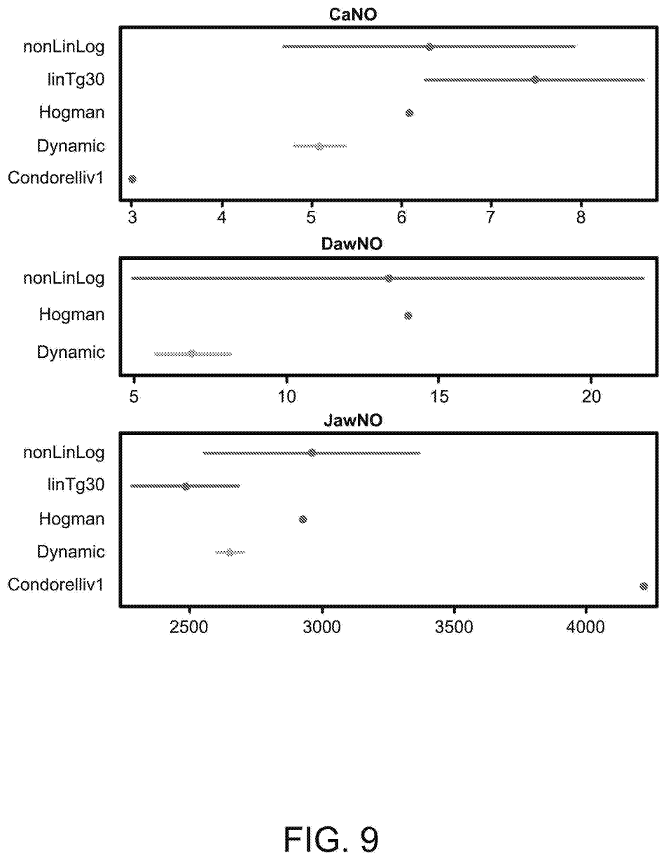

The lighter lines in FIG. 9 (e.g., line for Dynamic) illustrate the credible intervals appearing in the table of FIG. 7. The chains were run for 10,000 steps, with the first 10% discarded as “burn-in”. Additionally, a shorter, 2,000-step chain was run first. The results of the shorter chain were used to initialize the longer chains. The darker lines in FIG. 9 illustrate the point estimates and, where available, confidence intervals for these parameter values using a variety of current methods; see (2) for details about the other models.

While FIG. 9 indicates the dynamic approach has superior precision to competing methods, we do not claim these intervals capture all relevant sources of variation. Airway geometry plays an important role in the observed patterns of eNO, and possible extensions to the two compartment model appear in the discussion.

### Conservation Laws and Numerical Methods: Advection of NO in the Airway

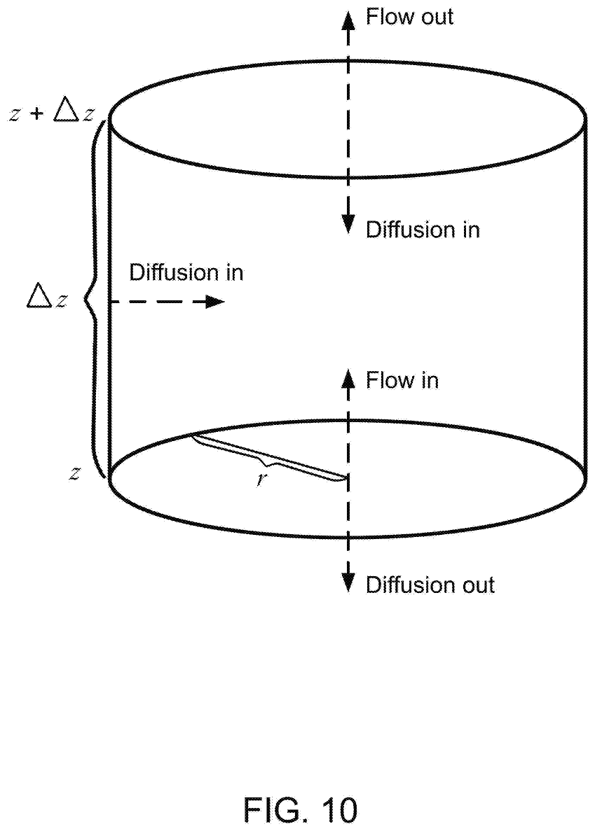

In some examples, the airway is assumed to be a cylinder with a fixed radius of rcm, a length of Lcm, and therefore the volume is V=πr² L; FIG. 10 illustrates a cross section of this cylinder with height Δz. c(t,z) denotes the concentration of NO at time t and at height z in

$$\frac{g}{{cm}^{3}} .$$

Although the general theory can accommodate variation in the other spatial dimensions (x,y), as the notation c(t,z) implies, one can assume the concentration depends only on the z (axial) coordinate.

### Airway Cross Section

During exhalation air moves vertically through the airway; therefore, air flows in through the lower boundary and out through the upper boundary. Denoting by v the velocity of gas through the airway in

$$\frac{cm}{s} ,$$

over the time interval (t,t+Δt) seconds a “slice” of air traveling at rate v will travel a distance vΔt=Δzcm. At time t the mass of NO in the region is approximately πr² Δzc(t,z), and at time t+Δt the mass is approximately πr² Δzc(t+Δt,z). Therefore, the change in mass over the time interval of length Δt is approximately Δm≈πr² Δz[c(t+Δt, z)−c(t,z)].

Over the time interval Δt, the mass that flows into the region through the lower boundary is approximately vΔtπr²c(t,z), and the mass that flows out of the region through the upper boundary is approximately vΔtπr²c (t, z+Δz). Therefore, the net change in mass due to flow (advection) is approximately vΔtπr²[c(t,z)−c(t, z+Δz)]=−vΔtπr²[c(t, z+Δz)−c(t,z)]

### Diffusion of NO in the Airway

According to Fick's first law, the diffusive flux will be proportional to the concentration gradient

$$\frac{\partial c}{\partial z} .$$

Denoting by d the proportionality constant (diffusivity of NO in air, in

$$\frac{{cm}^{2}}{s} ) ,$$

over the time interval Δt the mass diffusing into the region will be approximately

$$Δ t π {r}^{2} d \frac{\partial c}{\partial z} ( t , z + Δ z )$$

and the mass diffusing out of the region will be approximately

$$Δ t π {r}^{2} d \frac{\partial c}{\partial z} ( t , z ) .$$

Therefore, over the time interval Δt the net change in mass due to diffusion is approximately

$$Δ t π {r}^{2} d [ \frac{\partial c}{\partial z} ( t , z + Δ z ) - \frac{\partial c}{\partial z} ( t , z ) ] .$$

### Sources of NO in the Airway

There will also be a contribution of NO into the region by diffusion from the airway wall at a rate proportional to the concentration difference. Denoting by c_{w} the constant airway wall concentration, and p the coefficient of NO permeability from tissue to lumen, the net mass diffusing into the region over the time interval (t, t+Δt) from the airway wall is approximately Δt2πrΔzp[c_{w}−c (t,z)], i.e. the product of the time interval, the airway wall surface area, a constant coefficient, and the difference in concentration between the airway wall and lumen.

The principle of mass conservation indicates that the change in mass over the time interval (t, t+Δt) must be the sum of the net flows and diffusion across the boundary. This implies the approximate equality:

$$π {r}^{2} Δ z [ c ( t + Δ t , z ) - c ( t , z ) ] \approx - v Δ t π {r}^{2} [ c ( t , z + Δ z ) - c ( t , z ) ] + Δ t π {r}^{2} d [ \frac{\partial c}{\partial z} ( t , z + Δ z ) - \frac{\partial c}{\partial z} ( t , z ) ] + Δ t 2 π r Δ zp [ {c}_{w} - c ( t , z ) ] .$$

Dividing both sides by πr²ΔtΔz yields:

$$\frac{c ( t + Δ t , z ) - c ( t , z )}{Δ t} \approx - v \frac{c ( t , z + Δ z ) - c ( t , z )}{Δ z} + d \frac{\frac{\partial c}{\partial z} ( t , z + Δ z ) - \frac{\partial c}{\partial z} ( t , z )}{Δ z} + \frac{2 p}{r} [ {c}_{w} - c ( t , z ) ] ,$$

and letting Δt,Δz→0 results in the partial differential equation (1) or model (S255).

### Conservative Form and Numerical Methods

In the preceding derivation, the coefficients v and d multiplying the advective and diffusive terms (respectively) were assumed to be constant. If either of these quantities varies with time, that is v=v(t), d=d(t) the result is almost identical; essentially, the only change is that one or both of v(t), d(t) replaces the corresponding constant (as was done with v in). However, if either coefficient depends on the space (axial position) variable, that is v=v(z) and/or d=d(z), the result no longer holds.

To accommodate the general case, wherein v=v(t,z) and d=d(t,z) may be functions of both space and time, the problem can be reformulated as: where simplifies to when

$$f ( c ( t , z ) ) = v * c ( t , z ) - d * \frac{\partial }{\partial z} c ( t , z ) and h ( c ( t , z ) ) = \frac{2 p}{r} [ {c}_{w} - c ( t , z ) ] .$$

With different choices for the functions ƒ and h, it is possible to express other conservation laws, such as for energy and momentum, in the same form (7). An equation following the template of is said to be written in, and in this form ƒ is known as the.

In general, conservation laws, which can be expressed in the form, cannot be solved analytically; rather, this form has proven most useful as a basis for numerical methods (5, 7). Because initial and boundary conditions are crucial components of any numerical method, even simple extensions of model problems often require customized numerical algorithms. For example, simply extending the steady state two-compartment model to one where the flow rate is allowed to vary with time requires a custom written software solution.

### Boundary and Initial Conditions

For equation to have a unique solution, initial (time) and inflow boundary (space) conditions must be specified; although in general, it can only be calculated numerically. Moreover, the incoming concentration depends on the direction of flow. During exhalation it corresponds to the alveolar concentration, a parameter to be estimated. During inhalation this concentration is typically the ambient NO level; however, during testing subjects may be provided air that has been “scrubbed” of NO.

By definition, modeling FeNO involves modeling exhalation. However, because respiration is cyclic, the terminal condition in one direction of flow becomes the initial condition for the reverse flow. This relationship means that NO measured during exhalation is determined, in part, by the terminal state of the previous inhalation. In principle, the previous inhalation depends, in turn, on the preceding exhalation, which depends on the inhalation before that, continuing ad nauseam.

In practice, higher flow rates diminish this dependence, and at relatively high rates (300+ ml/s), the terminal airway concentration is effectively independent of the initial. Although 300 ml/s is a relatively rapid rate for exhalation, it is a relatively slow rate for inhalation. For example in all 74 maneuvers illustrated in the supplement, this threshold was cleared every time, typically by factors of at least 2-3×.

The implication of this phenomenon is that calculating accurate estimates of the airway concentration immediately after inhalation does not require knowing the initial airway concentration when inhalation began. The solution can be calculated based on a simple initial condition (i.e. zero everywhere), and the end result will be essentially unchanged. The terminal condition will also depend on the inflow concentration; however, in the case of “scrubbed” air this can be assumed to be zero.

### REFERENCES

1. Dormand J R, Prince P J. A family of embedded Runge-Kutta formulae. Journal of computational and applied mathematics 6: 19-26, 1980.
2. Eckel S P, Linn W S, Berhane K, Rappaport E B, Salam M T, Zhang Y, Gilliland F D. Estimation of parameters in the two-compartment model for exhaled nitric oxide. PLoS ONE 9: e85471, 2014.
3. George S C, Hogman M, Permutt S, Silkoff P E. Modeling pulmonary nitric oxide exchange. J Appl Physiol 96: 831-839, 2004.
4. Haario H, Saksman E, Tamminen J. An adaptive metropolis algorithm. Bernoulli.
5. Hundsdorfer W, Verwer J. Numerical solution of time-dependent advection-diffusion-reaction equations. Berlin: Springer, 2003.
6. III JOS. Introduction to digital filters: With audio applications. W3K Publishing, 2007.
7. LeVeque R J. Numerical methods for conservation laws. Basel: Springer Basel AG, 1992.
8. LeVeque R J. Finite difference methods for ordinary and partial differential equations: Steady-state and time-dependent problems. Philadelphia, Pa.: Society for Industrial; Applied Mathematics, 2007.
9. Roberts G O, Rosenthal J S. Examples of adaptive mCMC. Journal of Computational and Graphical Statistics 18: 349-367, 2009.
10. Shin H W, George S C. Impact of axial diffusion on nitric oxide exchange in the lungs. J Appl Physiol 93: 2070-2080, 2002.
11. Tsoukias N M, George S C. A two-compartment model of pulmonary nitric oxide exchange dynamics. J Appl Physiol 85: 653-666, 1998.
12. Weibel E R. Morphometry of the human lung. Springer, 1963.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system may include client, servers, communication network, and a database. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for determining a concentration of nitric oxide in exhaled breath, the system comprising:
a flow chamber;
a flow sensor connected to the flow chamber positioned to detect flow of gas inside the flow chamber;
a nitric oxide sensor positioned inside the flow chamber, the nitric oxide sensor positioned to detect a local nitric oxide concentration of gas inside the flow chamber;
a memory containing machine readable medium comprising machine executable code having stored thereon instructions for performing a method of determining an exhaled concentration of nitric oxide; and
a control system coupled to the memory, the control system configured to execute the machine executable code to cause the processor to:
receive, from the flow sensor, flow rate data points that include data related to a flow rate and a time stamp corresponding to the flow rate;
receive, from the nitric oxide sensor, concentration data points that comprise data related to a local nitric oxide concentration and a time stamp corresponding to the local nitric oxide concentration;
associate, by the control system, the flow rate data points and the concentration data points that both have a time stamp indicating they were taken within close or at same temporal proximity;
store, by the control system, associated flow rate data points and concentration data points for an exhale event; and
determine, by the control system, an indication of an exhaled nitric oxide concentration based on at least a subset of the stored associated flow rate data points and concentration data points for the exhale event, wherein the subset of stored associated flow rate data points comprises data related to different flow rates.

2. The system of claim 1, wherein the step of determining the indication of an exhaled nitric oxide concentration is performed using a differential equation to model the flow rate.

3. The system of claim 2, wherein the differential equation is an advection-diffusion-reaction partial differential equation.

4. The system of claim 1, wherein the flow rate data points by the flow sensor is output to a low pass filter.

5. The system of claim 3, wherein parameter estimates of the advection-diffusion-reaction partial differential equation are run by the control system using a Markov Chain.

6. The system of claim 1, wherein the flow sensor is selected from at least one of: a pressure differential sensor, an ultrasonic flow meter, an optical flow sensor, and a mechanical flow sensor.

7. The system of claim 1, wherein the nitric oxide sensor comprises an electrochemical sensor.

8. The system of claim 1, wherein the control system is embedded in a remote server or a database.

9. The system of claim 1, wherein the flow chamber comprises a mouthpiece.

10. A system for determining a concentration nitric oxide in exhaled breath in a patient, the system comprising:
a device that measures and transmits at least two output signals associated with the patient;
a computing device configured to receive, record, store, and analyze the at least two output signals from the device to generate the patient's concentration of nitric oxide in exhaled breath; and
a graphical user interface on the computing device that allows a user to view and customize options for monitoring the concentration of nitric oxide in exhaled breath,
wherein the device, the at least one remote device, and the computing device are communicatively connected to each other via a communications network,
wherein the device comprises a flow chamber, a flow sensor and a nitric oxide sensor,
wherein the at least two outputs signal comprises: (i) flow rate and a time stamp corresponding to the flow rate and (ii) a local nitric oxide concentration and a time stamp corresponding to the local nitric oxide concentration,
wherein the computing device receives, records, and stores associated flow rate data points and concentration data points for an exhale event of the patient, and
wherein the computing device is configured to determine an indication of an exhaled nitric oxide concentration based on at least a subset of the stored associated flow rate data points and concentration data points for the exhale event, wherein the subset of stored associated flow rate data points comprises data related to different flow rates.

11. The system of claim 10, wherein the system further comprises a hosted server that is (i) configured to store and analyze the at least two output signals, and (ii) connected to the device, the at least one remote device, and the computing device via the communications network.

12. The system of claim 11, wherein the hosted server is further configured to measure, store, and analyze the at least two output signals associated with a certain patient and dynamically aggregate and analyze the exhale event of the patient with at least two output signals to determine the concentration of nitric oxide in exhaled breath associated with the patient.

13. The system of claim 10, wherein the device is configured to send an alarm or a notification to the computing device or a healthcare professional based on a level of the concentration of nitric oxide in exhaled breath.

14. The system of claim 10, wherein the device is configured to detect a health event based on the at least two output signals and send an alert to at least one of following: the device, the computing device, or a healthcare professional.

15. The system of claim 10, wherein the step of determining the indication of an exhaled nitric oxide concentration is performed using a differential equation to model the flow rate.

16. The system of claim 15, wherein the differential equation is an advection-diffusion-reaction partial differential equation.

17. The system of claim 10, wherein the flow rate data points by the flow sensor is output to a low pass filter.

18. The system of claim 16, wherein parameter estimates of the advection-diffusion-reaction partial differential equation are run by the control system using a Markov Chain.

19. The system of claim 10, wherein the flow sensor is selected from at least one of: a pressure differential sensor, an ultrasonic flow meter, an optical flow sensor, and a mechanical flow sensor.

20. The system of claim 10, wherein the nitric oxide sensor comprises an electrochemical sensor.

21. The system of claim 10, wherein the flow chamber comprises a mouthpiece.
